(19) United States
(12) Patent Application Publication
(10) Pub. No.: US 2022/0054024 A1
(43) Pub. Date: Feb. 24, 2022

(54) SPHYGMOMANOMETER, METHOD FOR CONTROLLING SPHYGMOMANOMETER, AND METHOD FOR DETECTING EFFECTIVE PULSE WAVE

(71) Applicant: VITA-COURSE TECHNOLOGIES CO., LTD., Shenzhen (CN)

(72) Inventors: Rui LIU, Shenzhen (CN); Zhiyong WANG, Shenzhen (CN); Jiwei ZHAO, Shenzhen (CN); Chuanmin WEI, Shenzhen (CN)

(73) Assignee: VITA-COURSE TECHNOLOGIES CO., LTD., Shenzhen (CN)

(21) Appl. No.: 17/453,868

(22) Filed: Nov. 8, 2021

Related U.S. Application Data
(63) Continuation of application No. PCT/CN2019/086053, filed on May 8, 2019.

(51) Int. Cl. A61B 5/0225; A61B 5/021; A61B 5/00

(52) U.S. Cl. CPC A61B 5/0225; A61B 5/7203; A61B 5/7221; A61B 5/02116

(57) ABSTRACT

Embodiments of the present disclosure disclose a method for detecting an effective pulse wave and provide a sphygmomanometer and a method for controlling the sphygmomanometer. The method for detecting the effective pulse wave may include: selecting a pulse wave controlling parameter; setting an initial pulse wave controlling parameter; determining at least one pulse wave based on the initial pulse wave controlling parameter, determining a corrected initial pulse control parameter by correcting the initial pulse wave controlling parameter based on at least one pulse wave controlling parameter of the at least one pulse wave; determining at least one subsequent pulse wave based on the corrected initial pulse wave controlling parameter, and further correcting the corrected pulse wave controlling parameter based on at least one pulse wave controlling parameter of the at least one subsequent pulse wave; and repeating above iterative process and continuously correcting the initial pulse wave controlling parameter, and extracting at least one effective pulse wave based on the pulse wave controlling parameter after the correction. The present disclosure may be closer to an actual situation of a measured subject by correcting the initial pulse wave controlling parameter continuously, thereby filtering out a detected invalid pulse wave and avoiding missing detection of the effective pulse wave.

### CROSS-REFERENCE TO RELATED APPLICATIONS

This application is a continuation of International Patent Application No. PCT/CN2019/086053, filed on May 8, 2019, the contents of which are hereby incorporated by reference.

### TECHNICAL FIELD

The present disclosure relates to a medical device field, in particular, to a sphygmomanometer, a method for controlling the sphygmomanometer, and a method for detecting an effective pulse wave.

### BACKGROUND

A sphygmomanometer is a kind of widely used medical detection device. A systolic pressure/diastolic pressure of a person to be measured can be calculated by adjusting an air pressure in a cuff. In this manner, a pulse wave is the basis for accurately calculating the systolic pressure/diastolic pressure.

Therefore, it is necessary to provide a method for detecting an effective pulse wave and provide a sphygmomanometer and a method for controlling the sphygmomanometer.

### SUMMARY

One aspect of the present disclosure may provide a method of controlling a sphygmomanometer. The method may include: selecting a pulse wave controlling parameter, wherein the pulse wave controlling parameter includes at least one of an amplitude threshold, a time threshold, or a heart rate threshold; setting an initial pulse wave controlling parameter; performing pressurization of a sphygmomanometer; determining at least one pulse wave based on the initial pulse wave controlling parameter during the pressurization; determining a corrected initial pulse wave controlling parameter by correcting the initial pulse wave controlling parameter based on at least one pulse wave controlling parameter of the at least one pulse wave; determining at least one subsequent pulse wave based on the corrected pulse wave controlling parameter; further correcting the corrected initial pulse wave controlling parameter based on at least one pulse wave controlling parameter of the at least one subsequent pulse wave; repeating above iterative process and continuously correcting the initial pulse wave controlling parameter; extracting at least one effective pulse wave based on the initial pulse wave controlling parameter after the correction; and generating a blood pressure measurement result based on a detection result of the at least one effective pulse wave.

In some embodiments, the correcting the initial pulse wave controlling parameter may include: identifying a first pulse wave and a second pulse wave, wherein amplitudes of the first pulse wave and the second pulse wave are greater than an initial amplitude threshold; correcting the initial amplitude threshold based on the amplitude of the first pulse wave and the amplitude of the second pulse wave after an initial determination that the first pulse wave and the second pulse wave are eligible; identifying a third pulse wave based on the corrected initial amplitude threshold, an initial heart rate threshold, and/or an initial time threshold; further correcting the initial amplitude threshold based on an amplitude of the third pulse wave; correcting the initial heart rate threshold and/or the initial time threshold based on a time interval between the third pulse wave and the second pulse wave; and determining a subsequent pulse wave based on the corrected initial amplitude threshold, the corrected initial time threshold, and/or the corrected initial heart rate threshold.

In some embodiments, a condition for the initial determination that the first pulse wave and the second pulse wave are eligible may include that a time interval between the first pulse wave and the second pulse wave is within a range of the initial heart rate threshold and/or the initial time threshold; if the time interval between the first pulse wave and the second pulse wave is out of the range of the initial heart rate threshold or the initial time threshold, discarding the first pulse, and determining a subsequent pulse wave of an amplitude greater than the initial amplitude threshold until a time interval between two adjacent pulse waves is within the range of the initial heart rate threshold or the initial time threshold.

In some embodiments, the initial amplitude threshold may be a pressure of 0.2 mmHg; the initial heart rate threshold may be a minimum of 30 times per minute and a maximum of 300 times per minute; or the initial time threshold may be a minimum of 0.2 s and a maximum of 2 s.

In some embodiments, the method may further include generating an effective pulse wave template based on the at least one effective pulse wave; and filtering out noise interference in a pulse wave using the effective pulse wave template.

In some embodiments, if the noise interference is in a non-pulse wave portion of the effective pulse wave template, directly filtering out the noise interference; and if the noise interference is in a pulse wave portion of the effective pulse wave template, performing fitting and compensation on the pulse wave.

In some embodiments, the performing fitting and compensation may include determining a changing trend of the at least one effective pulse wave over time based on the effective pulse wave template; determining an inflection point in a curve of the changing trend; determining an amplitude of the pulse wave based on an average of a sum of an amplitude of an effective pulse wave before the pulse wave and an effective pulse wave after the pulse wave in the effective pulse wave template; determining a pulse wave time based on the effective pulse wave template; and determining the pulse wave based on the pulse wave time, the amplitude of the pulse wave, and a position of the inflection point by a curve fitting technique using ordinary least squares.

In some embodiments, the generating a blood pressure measurement result based on a detection result of the at least one effective pulse wave may include: determining an amplitude of a pulse wave based on the initial pulse wave controlling parameter after the correction by: determining a minimum value before the pulse wave; and designating a maximum value among a plurality of sample points after a start point as a maximum value of the pulse wave, the start point corresponding to a first maximum value after the minimum value.

In some embodiments, a count of the plurality of sampling points may relate to a heart rate, and a time interval of two adjacent sampling points of the plurality of sampling points may be 1 millisecond.

In some embodiments, the method may further include generating a final pressure for when the pressurization is to be ended based on the detection result of the at least one effective pulse wave, wherein the pressurization of the sphygmomanometer is ended if a pressure of the sphygmomanometer reaches the final pressure.

In some embodiments, the generating a final pressure may include: extracting an amplitude of each of the at least one effective pulse wave; comparing at least one rising or declining trend of at least one amplitude of the at least one effective pulse wave; determining a peak value of the at least one amplitude; and determining the final pressure according to formula below:

P=K×(Hr×m+Mp)

where K may be a linear correlation coefficient of the pressurization in a macroscopic scale, Hr may be a time interval between two adjacent effective pulse waves, m may be a systolic pressure coefficient, and Mp may be a time coordinate corresponding to the peak value of the at least one amplitude.

In some embodiments, the method may further include controlling a pressurization speed by: presetting a relationship between pressure and time, wherein each of pressures at different time points is determined based on the relationship and designated as a preset pressure, respectively; detecting an actual pressure of the sphygmomanometer during the pressurization; correcting the preset pressure based on the actual pressure; and adjusting the pressurization speed of the sphygmomanometer based on the corrected preset pressure.

In some embodiments, the adjusting the pressurization speed of the sphygmomanometer based on the corrected preset pressure may include: comparing the actual pressure with the preset pressure; if the actual pressure is greater than the preset pressure, decreasing the pressurization speed; if the actual pressure is smaller than the preset pressure, increasing the pressurization speed; or if the actual pressure is equal to the preset pressure, maintaining the pressurization speed.

In some embodiments, the increasing or decreasing the pressurization speed may relate to a difference between the actual pressure and the preset pressure.

In some embodiments, the pressurization speed may be adjusted using proportional adjustment in a proportional-integral-derivative (PID) control algorithm.

In some embodiments, the preset relationship between pressure and time may be a linear relationship.

A second aspect of the present disclosure may provide a sphygmomanometer. The sphygmomanometer may include: a cuff configured to wind around a region to be measured; an electric machine configured to inflate air into the cuff for pressurization; an initial parameter setting unit configured to select a pulse wave controlling parameter and set an initial pulse wave controlling parameter, wherein the pulse wave controlling parameter includes at least one of an amplitude threshold, a time threshold, or a heart rate threshold; a correction processing unit configured to continuously correct the initial pulse wave controlling parameter during the pressurization of the electric machine based on the initial pulse wave controlling parameter and a pulse wave determined based on the initial pulse wave controlling parameter, and extract at least one effective pulse wave based on the initial pulse wave controlling parameter after the correction; and a blood pressure determination unit configured to generate a blood pressure measurement result based on a detection result of the at least one effective pulse wave.

In some embodiments, the sphygmomanometer may further include a pressure detection component configured to detect an actual pressure inside the cuff during the pressurization; and an electric machine control component configured to adjust a pressurization speed of the electric machine. The electric machine control component may adjust the pressurization speed of the electric machine based on the actual pressure detected by the pressure detection component.

In some embodiments, the blood pressure determination unit may determine a signal for ending the pressurization based on the at least one effective pulse wave extracted by the correction processing unit, and determine a final pressure; and the electric machine control component may control the electric machine to perform the pressurization and stop the pressurization if a pressure reaches the final pressure.

A third aspect of the present disclosure may provide a method for detecting an effective pulse wave. The method may include selecting a pulse wave controlling parameter, wherein the pulse wave controlling parameter includes at least one of an amplitude threshold, a time threshold, or a heart rate threshold; setting an initial pulse wave controlling parameter; determining at least one pulse wave based on the initial pulse wave controlling parameter; determining a corrected initial pulse wave controlling parameter by correcting the initial pulse wave controlling parameter based on at least one pulse wave controlling parameter of the at least one pulse wave; determining at least one subsequent pulse wave based on the corrected initial pulse wave controlling parameter; further correcting the corrected initial pulse wave controlling parameter based on at least one pulse wave controlling parameter of the at least one subsequent pulse wave; repeating above iterative process and continuously correcting the initial pulse wave controlling parameter; and extracting at least one effective pulse wave based on the initial pulse wave controlling parameter after the correction.

### BRIEF DESCRIPTION OF THE DRAWINGS

In order to more clearly describe the technical solutions of the embodiments of the present disclosure, the drawings that need to be used in the description of the embodiments will be briefly introduced below. Obviously, the drawings described below are only some examples or embodiments of the present disclosure. For those of ordinary skill in the art, without creative work, the present disclosure can be applied to other similar scenarios according to these drawings, and wherein:

FIG. 1 is a block diagram illustrating an exemplary relationship between components of a sphygmomanometer according to some embodiments of the present disclosure;

FIG. 2 is a flowchart illustrating an exemplary process for a method of adjusting a pressurization speed of a sphygmomanometer based on a corrected preset pressure according to some embodiments of the present disclosure;

FIG. 3 is a flowchart illustrating an exemplary process for detecting an effective pulse wave according to some embodiments of the present disclosure;

FIG. 4 is a flowchart illustrating an exemplary process for controlling a sphygmomanometer according to some embodiments of the present disclosure; and

FIG. 5 is a schematic diagram illustrating an exemplary detection result of a deformed waveform due to noise interference according to some embodiments of the present disclosure.

### DETAILED DESCRIPTION

The flowcharts used in the present disclosure may illustrate operations executed by the system according to embodiments in the present disclosure. It should be understood that a previous operation or a subsequent operation of the flowcharts may not be accurately implemented in order. Conversely, various operations may be performed in inverted order, or simultaneously. Moreover, other operations may be added to the flowcharts, and one or more operations may be removed from the flowcharts.

It will be understood that the terms “device,” “component,” and/or “unit” used herein are one method to distinguish different components, elements, parts, sections, or assemblies of different levels. However, the terms may be displaced by another expression if they achieve the same purpose.

Although various references are made according to some components or units in the embodiments of the present disclosure, any count of different components or units may be used and run on a client and/or a server. The components or units are merely illustrative, and different components or units can be used in different aspects of the sphygmomanometer and the method.

As shown in the present disclosure and claims, unless the context clearly indicates exceptions, the words “a,” “an,” “one,” and/or “the” do not specifically refer to the singular, but may also include the plural. The terms “including” and “comprising” only suggest that the steps and elements that have been clearly identified are included, and these steps and elements do not constitute an exclusive list, and the method or device may also include other steps or elements.

Main terms in the present disclosure may be defined first. As used herein, a pulse wave amplitude is an amplitude of a pulse wave, that is, a pressure difference between a maximum value and a minimum value. A pulse wave time is an oscillation period of a complete pulse wave (e.g., a time interval between two adjacent crests of a wave). A heart rate is a count of heartbeats per minute. there is a conversion relationship between pulse wave time and heart rate, that is, 1/time interval=heart rate.

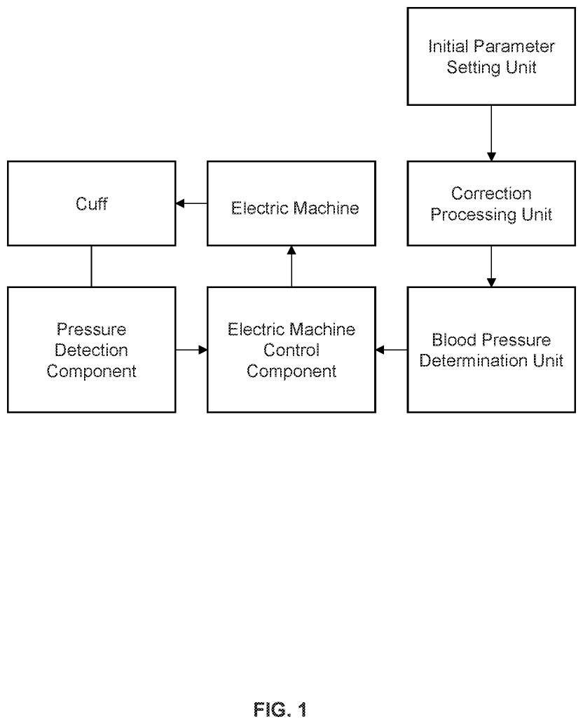

In one aspect of the present disclosure, a sphygmomanometer may be provided. FIG. 1 is a block diagram illustrating an exemplary relationship between components of a sphygmomanometer according to some embodiments of the present disclosure. For example, the sphygmomanometer may include a cuff configured to wind around a region to be measured, an electric machine configured to inflate air into the cuff for pressurization, an initial parameter setting unit configured to select a pulse wave controlling parameter and set an initial pulse wave controlling parameter, wherein the pulse wave controlling parameter includes at least one of an amplitude threshold, a time threshold, or a heart rate threshold; a correction processing unit configured to continuously correct the initial pulse wave controlling parameter in the pressurization of the electric machine based on the initial pulse wave controlling parameter and a pulse wave selected based on the initial pulse wave controlling parameter, and extract at least one effective pulse wave based on the initial pulse wave controlling parameter after the correction, and a blood pressure determination unit configured to generate a blood pressure measurement result based on a detection result of the at least one effective pulse wave.

A measurement principle and process of the sphygmomanometer provided in the present disclosure may include that: the electric machine is initiated, air is inflated into the cuff, a pressure in the cuff increases gradually, and a waveform of a pulse wave appears in the detection result at a certain moment (the certain moment corresponding to a moment that the vessel of a person to be measured is under pressure). The initial parameter setting unit, the correction processing unit, and the blood pressure determination unit may be configured to process the appeared pulse wave and determine a final blood pressure measurement result.

In some embodiments, the detection result may be obtained by a sensing module. In some embodiments, the sensing module may include one or more sensors. The sensor may be an external device, or a component or an electronic element of the external device. The sensing module may be one or more sensors integrated on a same electronic element, or a combination of a plurality of electronic elements (each of which including one or more sensors). Data types obtained by the sensing module may include but be not limited to: physical data, chemical data, biological data, etc. The physical data may include but be not limited to: sound, light, time, weight, proximity, position, temperature, humidity, pressure, current, velocity, acceleration, inhalable particle, radiation, text, image, touch, pupil, fingerprint, etc. The chemical data may include but be not limited to, air pollutant, water pollutant, carbon monoxide concentration, carbon dioxide concentration, etc. The biological data may include but be not limited to, blood pressure, heart rate, blood sugar, insulin, etc., of an organism. In some embodiments, a device used to detect and/or monitor sound may include but be not limited to, microphone, etc. In some embodiments, a device used to detect and/or monitor light may include but be not limited to, an illuminance sensor, an ambient light sensor, etc.

In some embodiments, the sphygmomanometer of the present disclosure may include a display module configured to display the blood pressure measurement result. Information output by the display module may include program, software, algorithm, data, text, digit, image, voice, etc., or any combination thereof.

It should be noted that the above descriptions are merely provided for the purposes of illustration, and not intended to limit the scope of the present disclosure. For person skilled in the art, after understanding the basic principle of the present disclosure, it is possible to make various modifications and changes in the form and details of the sphygmomanometer without departing from this principle. However, those variations and modifications do not depart from the scope of the present disclosure.

As still referred to FIG. 1, in some embodiments, the sphygmomanometer may further include a pressure detection component configured to detect an actual pressure in the cuff during the pressurization; an electric machine control component configured to adjust a pressurization speed of the electric machine. The electric machine control component may adjust the pressurization speed of the electric machine based on the actual pressure detected by the pressure detection component.

In some embodiments, the method for adjusting the pressurization speed may be: presetting a relationship between pressure and time, wherein each of pressures at different time points is determined based on the relationship and designated as a preset pressure, respectively, detecting an actual pressure of the sphygmomanometer during the pressurization, correcting the preset pressure based on the actual pressure, and adjusting the pressurization speed of the sphygmomanometer based on the corrected preset pressure. In some embodiments, the relationship may be a linear relationship. The purpose of adjusting the pressurization speed may be achieved if the relationship is nonlinear, however, a turning point may be shown in the relationship between pressure and time, and the turning point may be errored as a pulse wave in a process for collecting the pulse wave, causing interference and misjudgment on a signal of the pulse wave. Using linear pressurization may make sure that the pressurization process is stable, and a collected waveform is clean and reasonable.

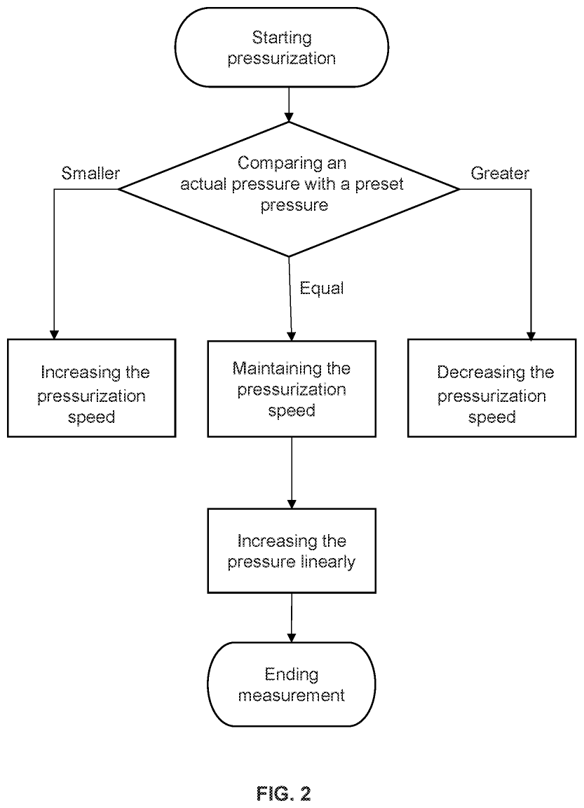

FIG. 2 is a flowchart illustrating an exemplary process for a method of adjusting a pressurization speed of a sphygmomanometer based on a corrected preset pressure after correction according to some embodiments of the present disclosure. In some embodiments, the detected actual pressure (the actual pressure being detected per 100 ms by the pressure sensor) may be compared with the preset pressure. If the actual pressure is greater than the preset pressure, the pressurization speed may be decreased; if the actual pressure is smaller than the preset pressure, the pressurization speed may be increased; if the actual pressure is equal to the preset pressure, the pressurization speed may be maintained. In some other embodiments, a pressure difference threshold may be set, such as setting a maximum pressure difference and a minimum pressure difference. If a difference between the actual pressure and the preset pressure is between the maximum pressure difference and the minimum pressure difference, the pressurization speed may be maintained; if the difference between the actual pressure and the preset pressure is smaller than the minimum pressure difference, the pressurization speed may be increased; if the difference between the actual pressure and the preset pressure is greater than the maximum pressure difference, the pressurization speed may be decreased. By means of the pressure difference threshold, the pressure may increase linearly, and interference and influence of the pressurization process on a subsequent waveform analysis may be further reduced.

In some embodiments, the increment or decrement of the pressurization speed may be a fixed value. In some other embodiments, the increment or decrement of the pressurization speed may relate to the difference between the actual pressure and the preset pressure. For example, the larger the difference between the actual pressure and the preset pressure is, the larger the increment or decrement of the pressurization speed may be, thereby reasonably controlling the pressurization speed.

In some embodiments, characteristics of the electric machine may be studied in the present disclosure based on a large amount of data. A data relationship between cuff and arm may be analyzed and fitted. Proportional adjustment in a PID (proportional-integral-derivative) control algorithm may be selected to adjust the pressurization speed. For example, in order to make the speed of the electric machine adjustable and reasonable, an initial speed of the electric machine may be not set too large or too small. It has been found that a reasonable initial duty cycle of the electric machine may be 80%±5% based on research data. By only utilizing the proportional adjustment in the PID control algorithm, on the one hand, the duty cycle of the electric machine may be adjusted in real-time and quickly, such that the pressure may increase linearly, and the acceleration and deceleration of the electric machine may not inhibit or amplify the effective pulse wave, ensuring that the effective pulse wave may not be deformed by the PID adjustment, and reducing influence of the speed adjustment of the electric machine on the subsequent waveform analysis.

As still referred to FIG. 1, in some embodiments, the sphygmomanometer may further include: the blood pressure determination unit configured to determine a signal for ending the pressurization based on the at least one effective pulse wave extracted by the correction processing unit and determine a final pressure, and the electric machine control component configured to control the electric machine to perform the pressurization and stop the pressurization if a pressure reaches the final pressure.

In some embodiments, the final pressure for when the pressurization is to be ended may be generated based on the detection result of the at least one effective pulse wave. The pressurization of the sphygmomanometer is ended if the pressure of the sphygmomanometer reaches the final pressure.

In some embodiments, a method for obtaining the final pressure may include: extracting an amplitude of each of the at least one effective pulse wave, comparing at least one rising or declining trend of at least one amplitude of the at least one effective pulse wave, determining a peak value of the at least one amplitude, and determining the final pressure according to formula below:

P=K×(Hr×m+Mp) Formula (1)

where K is a linear correlation coefficient of the pressurization in a macroscopic scale, such as a proportional coefficient in the PID control algorithm (e.g., a slope of a line connecting a point corresponding to a peak value of an amplitude and the origin), a range of which may be 0˜+∞. In order to avoid pressurizing too fast or too slow, in some embodiments, the range of K may be 10˜20. Hr is a time interval between two adjacent effective pulse waves, a range of which may be 0.2 s˜2 s. m is a systolic pressure coefficient, which is a multiple obtained based on an actual experience, and represents different values in the algorithm. For example, a recognition point near the systolic pressure may be calculated based on a value lower than m times of the peak amplitude of effective pulse wave. A range of m may be 0.5˜5. Mp is at least one time coordinate corresponding to at least one peak value of the at least one amplitude of the at least one effective pulse wave, a range of which may be 8 s˜15 s.

Taking a specific embodiment as an example, a method for determining the final pressure may be described. In the embodiment, the proportional coefficient in the PID control algorithm is 12, then K is 12. A time interval between each two adjacent effective pulse waves may be different. Hr is 0.85 s obtained by performing averaging calculation. m is 2. If the at least one peak value of the at least one amplitude of the at least one effective pulse wave appears at 11.65 s, the value of Mp may be 11.65 s. By bringing the above values into formula (1), a final pressure P=160.2 mmHg may be calculated, and a systolic pressure in the blood pressure measurement result may be 135 mmHg. Compared with a manner in prior art that the systolic pressure plus 30 mmHg˜40 mmHg is used as a signal for ending the pressurization, the pressurization in the embodiment may be ended when the pressure is 25 mmHg greater than the systolic pressure. On premise of not affecting a test result, on the one hand, a utilization rate of the electric machine may be improved, on the other hand, a detection time may be shortened, and an inflation pressure and an inflation time may be reasonable.

In another embodiment, K is 16, and Hr is calculated as 1.3 s. m is 0.9, and Mp is 9.25 s. By bringing the above value into the formula (1), the final pressure P=166.72 mmHg may be calculated, and the systolic pressure of the blood pressure measurement result may be 135 mmHg. In the embodiment, the pressurization may be ended when the pressure is 32 mmHg greater than the systolic pressure.

As can be seen from the above two embodiments, a method for determining the time of ending the pressurization in the present disclosure may not require fitting interpolation, etc., of the amplitude of the pulse wave, thereby accurately ending the pressurization when the pressure is at least 10 mmHg greater than the systolic pressure.

It should be understood that the sphygmomanometer and its modules shown in FIG. 1 may be implemented in various ways. For example, in some embodiments, the device and its modules may be implemented by hardware, software, or a combination of software and hardware. As used herein, a hardware part may be implemented by using special logic; a software part may be stored in a storage and executed by an appropriate instruction executing system, for example, executed by a microprocessor or specially designed hardware. Those skilled in the art may understand that the above-mentioned method and system may be implemented by using computer-executable instructions and/or by being included in processor control codes, for example, on a carrier medium such as a magnetic disk, CD or DVD-ROM, a programmable storage such as a read-only memory (firmware) or a data carrier such as an optical or electronic signal carrier, which provides the code. The structure and its modules of the present disclosure may be not only implemented by a hardware circuit such as a super-large-scale integrated circuit or a gate array, a semiconductor such as a logic chip, a transistor, a programmable hardware device such as a field programmable gate array, a programmable logic device, etc., but also implemented by software executed by various types of processors, or implemented by a combination (e.g., firmware) of the above hardware circuit and software.

It should be noted that the above descriptions about structures and modules are only for convenience of descriptions, and do not limit the present disclosure within a scope of the mentioned embodiments. It may be understood that, for those skilled in the art, after understanding the principle of the structures, it is possible to arbitrarily combine various modules, or form sub devices to connect with other modules without departing from the scope of the present disclosure.

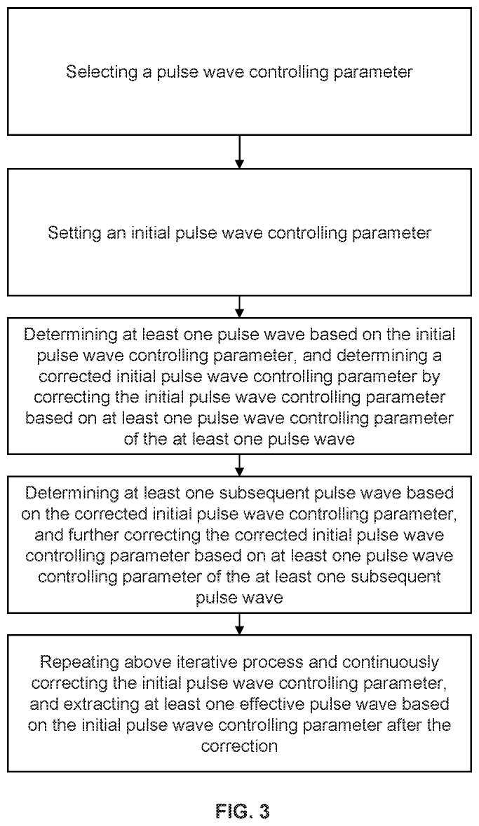

FIG. 3 is a flowchart illustrating an exemplary process for detecting an effective pulse wave according to some embodiments of the present disclosure. In another aspect of the present disclosure, a method for detecting an effective pulse wave is provided. The method may include: selecting a pulse wave controlling parameter, wherein the pulse wave controlling parameter includes at least one of an amplitude threshold, a time threshold, and/or a heart rate threshold; setting an initial pulse wave controlling parameter; determining at least one pulse wave based on the initial pulse wave controlling parameter, determining a corrected pulse wave controlling parameter by correcting the initial pulse wave controlling parameter based on at least one pulse wave controlling parameter of the at least one pulse wave, determining at least one subsequent pulse wave based on the corrected pulse wave controlling parameter, correcting the corrected pulse wave controlling parameter based on at least one pulse wave controlling parameter of the at least one subsequent pulse wave, repeating above iterative process and continuously correcting the initial pulse wave controlling parameter, and extracting at least one effective pulse wave based on the initial pulse wave controlling parameter after the correction.

In some embodiments, the pulse wave controlling parameter may include at least one of an amplitude threshold, a time threshold, and/or a heart rate threshold. It should be noted that the selection of the pulse wave controlling parameter may be related to an actual application scenario. In some embodiments, a pulse wave controlling parameter easily to be measured may be selected. In some other embodiments, for example, a heart rate of the subject to be measured may be abnormal due to nervousness or fear, and the heart rate threshold may be discarded as the pulse wave controlling parameter, and the amplitude threshold and the time threshold may be selected as the pulse wave controlling parameter. In some embodiments, the amplitude threshold and the heart rate threshold may be selected as the pulse wave controlling parameter. In some other embodiments, for example, differences between a plurality of measurement results may be relatively great, the pulse wave controlling parameter may include other thresholds (e.g., a ratio of a crest and a trough of the pulse wave) related to the pulse wave in addition to the amplitude threshold, the heart rate threshold, and/or time threshold, to improve measurement accuracy.

The initial pulse wave controlling parameter may be set. In some embodiments, an initial amplitude threshold may be greater than 0.1 mmHg, preferably 0.20 mmHg˜0.50 mmHg, such as 0.2 mmHg. An initial heart rate threshold may be 10 times per minute to 350 times per minute, preferably 20 times per minute˜320 times per minute, such as a minimum of 30 times per minute and a maximum of 300 times per minute. An initial time threshold may be 0.17 s˜6 s, preferably 0.19 s˜3 s, such as a minimum of 0.2 s and a maximum of 2 s. A signal of the pulse wave in an initial stage may be detected in a relatively wide range as mentioned above to avoid missing a signal.

An execution component (e.g., an electric machine) may perform pressurization of the sphygmomanometer, such that a pressure detected by the sphygmomanometer may change to perform a blood pressure measurement.

At least one pulse wave may be selected based on the initial pulse wave controlling parameter during the pressurization, a corrected pulse wave controlling parameter may be determined by correcting the initial pulse wave controlling parameter based on at least one pulse wave controlling parameter of the at least one pulse wave. At least one subsequent pulse wave may be determined based on the corrected pulse wave controlling parameter, and the corrected pulse wave controlling parameter may be further corrected based on at least one pulse wave controlling parameter of the at least one subsequent pulse wave. The above iterative process may be repeated to continuously correct the initial pulse wave controlling parameter until the pressurization process is ended. At least one effective pulse wave may be extracted based on the pulse wave controlling parameter after the correction (a method for determining the effective pulse wave comprising generating an effective pulse wave template based on the initial pulse wave controlling parameter after the correction, for example, an amplitude threshold of the effective pulse wave template being 120 mmHg, a time threshold of the effective pulse wave template being 0.15 s, if a detected pulse wave is matched to the effective pulse wave template, the detected pulse wave may be designated as an effective pulse wave, otherwise the detected pulse wave may be not designated as an effective pulse wave); generating a blood pressure measurement result based on a detection result of the at least one effective pulse wave.

In the present disclosure, the pulse wave controlling parameter may be closer to a real situation of the subject through continuous correction. The at least one effective pulse wave extracted based on the initial pulse wave controlling parameter after the correction may filter out an invalid pulse wave caused by a too large or too small pulse wave controlling parameter, and also avoid missing detection of the effective pulse wave. Thus, the obtained effective pulse wave may be more integrated and accurate.

In some embodiments, a first pulse wave and a second pulse wave whose amplitudes are greater than the initial amplitude threshold (e.g., 0.2 mmHg) may be identified. The first pulse wave and the second pulse wave may be invalid, and an initial determination may be performed on the first pulse wave and the second pulse wave. A condition for the initial determination that the first pulse wave and the second pulse wave are eligible may include that a time interval between the first pulse wave and the second pulse wave is within a range of the initial heart rate threshold and/or the initial time threshold. If the time interval between the first pulse wave and the second pulse wave is out of the range of the initial heart rate threshold or the initial time threshold, the first pulse may be discarded, and a subsequent pulse wave of an amplitude greater than the initial amplitude threshold may be determined until a time interval between two adjacent pulse waves is within the range of the initial heart rate threshold or the initial time threshold. An initial filtration may be performed on the first pulse wave and the second pulse wave through the initial determination, and a portion of invalid pulse waves may be filtered out. After the initial determination is eligible, the initial amplitude threshold may be corrected based on the amplitude of the first pulse wave and the amplitude of the second pulse wave. A third pulse wave may be identified based on the corrected initial amplitude threshold, the initial heart rate threshold, and/or the initial time threshold. The initial amplitude threshold may further be corrected based on an amplitude of the third pulse wave. The initial heart rate threshold and/or the initial time threshold may be corrected based on a time interval between the third pulse wave and the second pulse wave. A subsequent pulse wave may be determined based on the corrected initial amplitude threshold, the corrected initial time threshold, and/or the corrected initial heart rate threshold.

Taking a specific detection result (an amplitude of the first pulse wave being 8 mmHg, an amplitude of the second pulse wave being 10 mmHg) as an example, a specific method for correcting the initial amplitude threshold based on the amplitude of the first pulse wave and the amplitude of the second pulse wave may be described below. In some embodiments, for a blood pressure measurement process of a patient with persistent hypertension, the method for correcting the amplitude threshold may be that the amplitude threshold may be corrected as one of the amplitude of the first pulse wave and the amplitude of the second pulse wave, which is closer to the initial amplitude threshold (e.g., 0.2 mmHg). In this embodiment, the amplitude threshold may be corrected as 8 mmHg. In some other embodiments, the method for correcting the amplitude threshold may be that the amplitude threshold may be corrected as an average of the amplitude of the first pulse wave and the amplitude of the second pulse wave. In the embodiment, the amplitude threshold may be corrected as 9 mmHg. In some other embodiments, for a blood pressure measurement process of a subject whose blood pressure is unstable, the method for correcting the amplitude threshold may be that: if the amplitude of the first pulse wave and the amplitude of the second pulse wave are both greater than the initial amplitude threshold, the amplitude threshold may be corrected as “the initial amplitude threshold+X” (X being a fixed value between 0.01 mmHg and 10 mmHg); if the amplitude of the first pulse wave and the amplitude of the second pulse wave are both smaller than the initial amplitude threshold, the amplitude threshold may be corrected as “initial amplitude threshold−X”; if one of the amplitude of the first pulse wave and the amplitude of the second pulse wave is greater than the initial amplitude threshold, and another is smaller than the initial amplitude threshold, the initial amplitude threshold may be maintained. In the embodiment, assuming that X is 2 mmHg, the amplitude threshold may be corrected as 2.2 mmHg.

Similarly, taking a specific measurement result (e.g., the detection result being 12 mmHg) as an example, a method for further correcting the initial amplitude threshold (the corrected initial amplitude threshold obtained in the above operation being 9 mmHg) based on the third pulse wave may be described. In some embodiments, if a difference between the amplitude of the third pulse wave and the corrected amplitude threshold obtained in the above operation is smaller than or equal to Y (Y being a fixed value between 0.01 mmHg and 5 mmHg), the corrected amplitude threshold obtained in the above operation may be not be corrected; if the difference between the amplitude of the third pulse wave and the corrected amplitude threshold obtained in the above operation is greater than Y, the corrected amplitude threshold obtained in the above operation may be corrected as “corrected amplitude threshold obtained in the above operation+Z” (Z being a fixed value between 0.01 mmHg and 1 mmHg). In the embodiment, Y is 2 mmHg and Z is 0.2 mmHg, and the corrected amplitude threshold obtained in the above operation may be further corrected as 9.2 mmHg. The method for subsequently correcting the initial amplitude threshold may refer to the above method and not be repeated herein.

Based on the time interval between the third pulse wave and the second pulse wave (e.g., 1.6 s), a specific method for correcting the initial heart rate threshold (including a minimum of 30 times per minute and a maximum of 300 times per minute) and/or the initial time threshold (including a minimum of 0.2 s and a maximum of 2 s) may be that, in some embodiments, the minimum or the maximum of the initial time threshold may be corrected as a time interval detected at present. Whether to correct the minimum or the maximum may depend on a difference between the time interval detected at present and the maximum, and a difference between the time interval detected at present and the minimum, the differences may be compared, and the maximum or the minimum that corresponds to the smaller difference may be corrected as the time interval detected at present. If the differences are equal, the minimum or maximum of the initial time threshold may not be corrected. In other words, one of two end points of the initial time threshold that is closer to the time interval detected at present may be corrected as the time interval detected at present. In this embodiment, the initial time threshold may be corrected to include the minimum of 0.2 s and the maximum of 1.6 s. In some other embodiments, one of the two end points of the initial time threshold that is closer to the time interval detected at present may be corrected. If the minimum is closer, the minimum may be corrected as “minimum+X” (where X is a fixed value between 0.01 s to 0.1 s); if the maximum is closer, the maximum may be corrected as “maximum−X”; if distances are equal, the minimum may be corrected as “minimum+X,” and the maximum may be corrected as “maximum−X;” or the maximum or minimum may not be corrected. In the embodiment, X is 0.05 s, and the initial time threshold may be corrected to include the minimum of 0.25 s or the maximum of 2 s. It should be noted that due to a conversion relationship between interval time and heart rate, that is, 1/time interval=heart rate, the heart rate threshold and the time threshold may be mutually converted, and the correction method may be the same. Thus, the methods for correcting the heart rate threshold, the subsequent heart rate threshold and the subsequent time threshold may refer to the method described above, which is not repeated herein.

It should be understood that the method shown in FIG. 3 may be implemented in various ways. For example, in some embodiments, the method may be implemented by hardware, software, or a combination of software and hardware. As used herein, a hardware part may be implemented by using special logic; a software part may be stored in a storage and executed by an appropriate instruction executing system, for example, executed by a microprocessor or specially designed hardware. Those skilled in the art may understand that the above-mentioned method and system may be implemented by using computer-executable instructions and/or by being included in processor control codes, for example, on a carrier medium such as a magnetic disk, CD or DVD-ROM, a programmable storage such as a read-only memory (firmware) or a data carrier such as an optical or electronic signal carrier, which provides the code. The method of the present disclosure may be not only implemented by a hardware circuit such as a super-large-scale integrated circuit or a gate array, a semiconductor such as a logic chip, a transistor, or a programmable hardware device such as a field programmable gate array, a programmable logic device, etc., but also implemented by software executed by various types of processors, or implemented by a combination (e.g., firmware) of the above hardware circuit and software.

It should be noted that the above descriptions of the method are only for convenience of descriptions, and do not limit the present disclosure within a scope of the mentioned embodiments. It may be understood that for those skilled in the art, after understanding the principle of the method, it is possible to arbitrarily combine various steps without departing from the scope of the present disclosure.

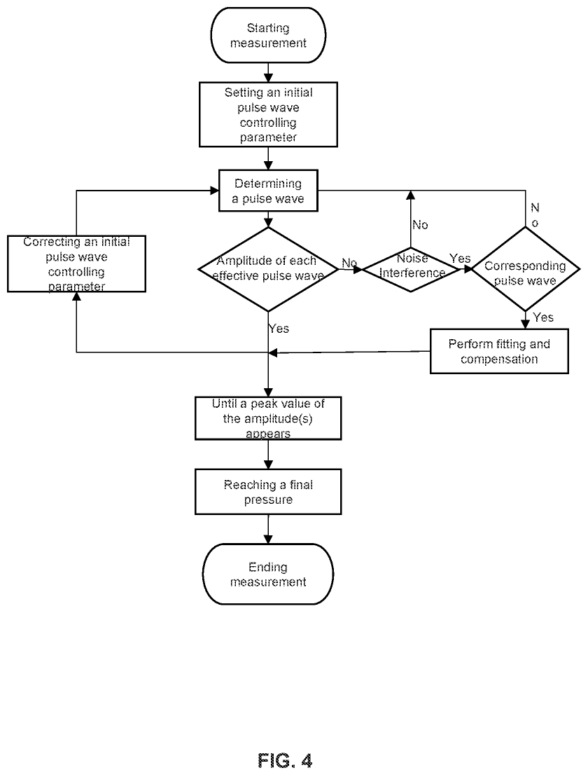

Another aspect of the present disclosure discloses a method for controlling a sphygmomanometer. FIG. 4 is a flowchart illustrating an exemplary process for controlling a sphygmomanometer according to some embodiments of the present disclosure. The exemplary process may include the following. The sphygmomanometer may begin to be inflated and a pressure in the sphygmomanometer may be gradually increased. A pressurization speed may be adjusted during the whole pressurization process, such that an actual pressurization speed may approximate a preset pressure. The pressurization process may be ended if a signal for ending the pressurization is detected by the sphygmomanometer. At a certain moment (corresponding to a time point when a blood vessel of the subject begins to be pressured) during the pressurization, a waveform of pulse waves may appear in a detection result. At least one effective pulse wave during the pressurization may be extracted. An amplitude of each effective pulse wave may be determined. Rising or declining trends of all amplitudes may be compared, and a peak value of the amplitudes may be determined and a final blood pressure measurement result may be determined.

It should be noted that the method for extracting the at least one effective pulse wave, adjusting the pressurization speed of the electric machine and determining the final blood pressure measurement result may be referred to the descriptions described above, and not be repeated herein.

In some embodiments, the sphygmomanometer may be disturbed by noise interference during the pressurization, causing incorrection recognition or missing recognition on a pulse wave or a sample point of the pulse wave, resulting in a deviation of a waveform signal, which is an important reason for the inaccurate blood pressure measurement result. Therefore, in some embodiments, in order to quickly obtain an accurate and integrated pulse waveform, the present disclosure provides a method for filtering out the noise interference in the pulse wave. The noise interference to be filtered out may include, but be not limited to, electromagnetic interference of a surrounding environment, conscious limb movement and unconscious muscle movement of the subject to be measured during measurement. However, a continuous strong amplitude interference may be not considered.

A specific method for filtering out the noise interference in the pulse wave may include generating an effective pulse wave template based on the at least one effective pulse wave, and filtering out the noise interference in the pulse wave using the effective pulse wave template.

As still referred to FIG. 4, in some embodiments, if the noise interference is in a non-pulse wave portion of the effective pulse wave template (e.g., a difference existing between a time interval between a detected pulse wave and an effective pulse wave before the pulse wave and a time interval obtained in the effective pulse wave template), the noise interference may be filtered out directly, and a subsequent effective pulse wave may be determined; if the noise interference is in a pulse wave portion of the effective pulse wave template (e.g., a time interval between a detected pulse wave and an effective pulse wave before the pulse wave matching a time interval obtained in the effective pulse wave template, however, an amplitude of the detected pulse wave being different from an amplitude obtained in the effective pulse wave template), an original pulse wave at the position may have been covered by the noise interference. A waveform at the position may not be filtered, and an original waveform may not be restored. Fitting and compensation may be performed on the pulse wave to compensate for a missing pulse wave at the position, thereby ensuring the integrity of the pulse wave obtained in the pressurization process.

In some embodiments, a method for the fitting and compensation may include: determining a changing trend of the at least one effective pulse wave over time based on the effective pulse wave template, determining an inflection point in a curve of the changing trend; determining an amplitude of the pulse wave based on an average value of a sum of an amplitude of an effective pulse wave before the pulse wave and an effective pulse wave after the pulse wave in the effective pulse wave template; determining a pulse wave time based on the effective pulse wave template; and determining the pulse wave based on the pulse wave time, the amplitude of the pulse wave, and a position of the inflection point by a curve fitting technique using an ordinary least squares.

The method for determining the pulse wave in the inflection point may be specifically described as follows:

Assuming that an n-th effective pulse wave before the noise interference is Nₖ₋ₙ, an amplitude of which is Aₖ₋ₙ. An n-th effective pulse wave after a position of noise interference of an effective pulse wave Nₖ with an amplitude Aₖ may be determined. Without considering continuous strong interference, an n-th effective pulse Nₖ₊ₙ with an amplitude Aₖ₊ₙ may be determined.

If noise interference appears at a first pulse wave, there may be no previous effective pulse wave, and the first pulse wave may be discarded directly.

If noise interference appears at top three sample points of a pulse wave, and

$${A}_{k + 1} > {A}_{k - 1} , {A}_{k} = \frac{{A}_{k + 1} + {A}_{k - 1}}{2} ,$$

otherwise, the measurement fails, and the measurement may be ended.

If noise interference appears after the third sample point, an increment of a previous effective pulse wave may be calculated. A weighted summation and an average may be performed on a difference d₁₂ between an amplitude of the first sample point before the noise interference and an amplitude of the second sample point, and a difference d₂₃ between the amplitude of the second sample point and an amplitude of the third sample point according to α and β, that is:

dₚ=(α×d₁₂+β×d₂₃)/(α+β)

Where dₚ is a predicted amplitude increment, α, β are correlation coefficients, which are determined according to an actual situation.

If Aₖ₊₁>Aₖ₋₁ and Aₖ₊₁≥Aₖ₋₁+2×dₚ,

$${A}_{k} = \frac{{A}_{k + 1} + {A}_{k - 1}}{2} .$$

Otherwise, if Aₖ₊₁>Aₖ₋₁ and Aₖ₊₁≥Aₖ₋₁+dₚ, Aₖ=Aₖ₋₁+δ×dₚ, otherwise, Aₖ=Aₖ₋₁+ε×dₚ.

Where δ and ε are amplitude correlation coefficients, which are determined according to an actual situation.

If Aₖ₊₁<Aₖ₋₁ and Aₖ₊₁≤Aₖ₋₁−2×dₚ,

$${A}_{k} = \frac{{A}_{k + 1} + {A}_{k - 1}}{2} .$$

Otherwise, if Aₖ₊₁<Aₖ₋₁ and Aₖ₊₁; Aₖ₋₁+dₚ, Aₖ=Aₖ₋₁−δ′×dₚ, otherwise Aₖ=Aₖ₋₁−ε′×dₚ.

Where δ′ and ε′ are amplitude correlation coefficients, which are determined according to an actual situation.

If Aₖ₊₁=Aₖ₋₁, Aₖ=Aₖ₋₁+ε″×dₚ.

Where ε″ is an amplitude correlation coefficient, which is determined according to an actual situation.

The amplitude corresponding to the noise interference may be determined according to the above-mentioned method. The inflection point in the curve may be determined (e.g., designating a position of a zero-crossing point of the curve of the changing trend obtained by one-time derivation as the inflection point) based on the pulse wave time and the changing trend of the at least one effective pulse wave over time determined in the effective pulse wave template. The pulse wave may be determined based on the pulse wave time, the amplitude of the pulse wave, and a position of the inflection point by a curve fitting technique using ordinary least squares to achieve compensation for the pulse wave.

The pulse wave after compensation may form an accurate and complete pulse waveform signal with the original effective pulse wave, avoiding inaccurate calculation of blood pressure measurement due to excessive pulse waves or inaccurate pulse waves caused by noise interference.

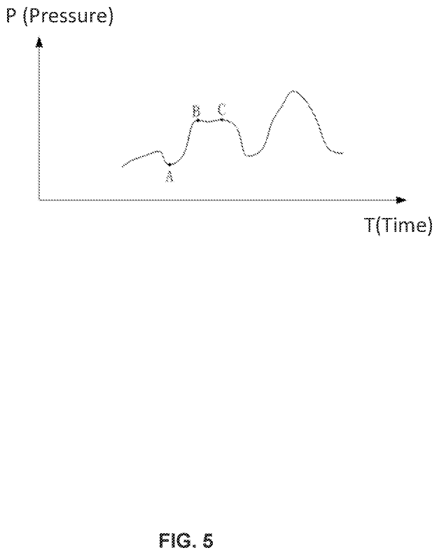

FIG. 5 is a schematic diagram illustrating an exemplary detection result of a waveform according to some embodiments of the present disclosure. The waveform may include two approximate maximum values in a same crest of a pulse wave due to noise interference. In some embodiments of the present disclosure, a minimum value (point A) before the pulse wave may be determined first, and then a maximum value among a plurality of sample points after a start point may be designated as a maximum value of the pulse wave. The start point may correspond to a first maximum value (point B) after the minimum value. In some embodiments, a time interval between two adjacent sample points may be 1 ms. In addition, a count of sample points may be related to a heart rate. For example, a time for collecting the sample points of the count may be smaller than a time interval between two pulse waves of most human beings. In this embodiment, the count of sample points is set as 200. In this way, a second maximum value (point C) among 200 sample points after the start point B may appear. At the time, pressures of point B and point C may be compared. In the embodiment, the pressure of point C is greater than the pressure of point B, point C may be selected as the maximum value of the pulse wave. Since a time interval between the two pulse waves of most human beings is greater than 0.2 s, the present disclosure may set 200 sample points, not only avoiding that crests in two adjacent pulse waves are errored as two sample points in a same waveform, but also covering situations of the two maximum caused by noise interference as shown in FIG. 5 to the largest extent. In the embodiment, the first maximum value in the pulse wave may be not selected, but the maximum value within a certain range may be selected as the maximum value, avoiding instability of the waveform caused by the noise interference, which causes the difficulty in determining a position of the maximum value.

Since the pressure may rise rapidly during the pressurization, if a signal is not quickly processed in time, the pressure may be over-pressured or under-pressured. Therefore, the present disclosure may control the pressurization process of the sphygmomanometer, for example, control the pressurization speed and a time for ending the pressurization.

The pressurization speed may be controlled. In some embodiments, a method for adjusting the pressurization speed may include: presetting a relationship between pressure over time, wherein each of pressures at different time points is determined based on the relationship and designated as a preset pressure, respectively; detecting an actual pressure of the sphygmomanometer during the pressurization; correcting the preset pressure based on the actual pressure; and adjusting the pressurization speed of the sphygmomanometer based on the corrected preset pressure. In some embodiments, the preset relationship between pressure and time may be a linear relationship. The purpose for adjusting the pressurization speed may be achieved if the relationship is nonlinear, however, a turning point may be shown in the relationship between pressure and time, and the turning point may be errored as a pulse wave in a process for collecting the pulse wave, causing interference and misjudgment on a signal of the pulse wave. Using linear pressurization may make sure that the pressurization process is stable, and a collected waveform is clean and reasonable.

In some embodiments, a method for correcting the preset pressure value based on the actual pressure, and a method for adjusting the pressurization speed of the sphygmomanometer based on the corrected preset pressure are similar to the methods of the above-mentioned descriptions, and not be repeated herein.

The time for ending the pressurization may be controlled. In some embodiments, a final pressure for when the pressurization is to be ended may be generated based on the detection result of the at least one effective pulse wave. The pressurization of the sphygmomanometer may be ended if a pressure of the sphygmomanometer reaches the final pressure.

A method for generating the final pressure may refer to the above-mentioned descriptions, and not be repeated herein.

It should be understood that the method shown in FIG. 4 may be implemented in various ways. For example, in some embodiments, the method may be implemented by hardware, software, or a combination of software and hardware. As used herein, a hardware part may be implemented by using special logic; a software part may be stored in a storage and executed by an appropriate instruction executing system, for example, executed by a microprocessor or specially designed hardware. Those skilled in the art may understand that the above-mentioned method and system may be implemented by using computer-executable instructions and/or by being included in processor control codes, for example, on a carrier medium such as a magnetic disk, CD or DVD-ROM, a programmable memory such as a read-only storage (firmware) or a data carrier such as an optical or electronic signal carrier, which provides the code. The controlling method of the present disclosure may be not only implemented by a hardware circuit such as a super-large-scale integrated circuit or a gate array, a semiconductor such as a logic chip, and a transistor, or a programmable hardware device such as a field programmable gate array, and a programmable logic device, etc., but also implemented by software executed by various types of processors, or may implemented by a combination (e.g., firmware) of the above hardware circuit and software.

It should be noted that the above descriptions of the controlling method are only for convenience of descriptions, and do not limit the present disclosure within a scope of the mentioned embodiments. It may be understood that for those skilled in the art, after understanding the principle of the system, it is possible to arbitrarily combine various modules. For example, in some embodiments, the method for filtering out the noise interference, the method for determining the effective pulse wave, the method for determining the final pressure, and the method for correcting the pulse wave controlling parameter may be used alone or in any combination. However, those variations do not depart from the scope of the present disclosure.

In another aspect of the present disclosure, a method for determining a signal for ending pressurization of the sphygmomanometer may be provided. The method may include: extracting an amplitude of each of at least one effective pulse wave; comparing at least one rising or declining trend of at least one amplitude of the at least one effective pulse wave; determining a peak value of the at least one amplitude; and determining the final pressure according to formula below:

P=K×(Hr×m+Mp)

Where K is a linear correlation coefficient of the pressurization in a macroscopic scale, Hr is a time interval between two adjacent effective pulse waves, m is a systolic pressure coefficient, and Mp is a time coordinate corresponding to the peak value of the at least one amplitude. The pressurization of the sphygmomanometer may be ended if a pressure of the sphygmomanometer reaches a final pressure. The principle and method of determining the final pressure value are similar to the above-mentioned descriptions, and may not be repeated herein.

In another aspect of the present disclosure, a method for filtering out noise interference in a pulse wave may include: generating an effective pulse wave template based on at least one effective pulse wave; and filtering out noise interference in the pulse wave using the effective pulse wave template. The specific method for filtering out the noise interference in the pulse wave using the effective pulse wave template may refer to the above-mentioned descriptions, and details may not be repeated herein. It should be noted that the method for detecting the at least one effective pulse wave may include but be not limited to the method provided in the present disclosure.

In another aspect of the present disclosure, a method for performing pressurization of a sphygmomanometer may include: presetting a relationship between pressure and time, wherein each of pressures at different time points is determined based on the relationship and designated as a preset pressure, respectively; detecting an actual pressure of the sphygmomanometer during the pressurization; correcting the preset pressure based on the actual pressure; and adjusting a pressurization speed of the sphygmomanometer based on the corrected preset pressure. The principle and method for correcting the preset pressure based on the actual pressure, and the principle and method for adjusting the pressurization speed of the sphygmomanometer based on the corrected preset pressure may be similar to the above-mentioned descriptions, and details may not be repeated herein.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “block,” “module,” “engine,” “unit,” “component,” or “system.” Furthermore, aspects of the present disclosure may take the form of a computer program product embodied in one or more computer-readable media having computer readable program code embodied thereon.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in claim. Rather, claim subject matter lies in less than all features of a single foregoing disclosed embodiment.

In some embodiments, the numbers expressing quantities of ingredients, properties, and so forth, used to describe and claim certain embodiments of the application are to be understood as being modified in some instances by the term “about,” “approximate,” or “substantially”. Unless otherwise stated, “about,” “approximate,” or “substantially” may indicate ±20% variation of the value it describes. Accordingly, in some embodiments, the numerical parameters set forth in the description and attached claims are approximations that may vary depending upon the desired properties sought to be obtained by a particular embodiment. In some embodiments, the numerical parameters should consider specified significant digits and adopt ordinary rounding techniques. Notwithstanding that the numerical ranges and parameters configured to illustrate the broad scope of some embodiments of the present disclosure are approximations, the numerical values in specific examples may be as accurate as possible within a practical scope.

Contents of each of patents, patent applications, publications of patent applications, and other materials, such as articles, books, specifications, publications, documents, etc., referenced herein are hereby incorporated by reference, excepting any prosecution file history that is inconsistent with or in conflict with the present document, or any file (now or later associated with the present disclosure) that may have a limiting effect to the broadest scope of the claims. It should be noted that if the description, definition, and/or terms used in the appended materials of the present disclosure is inconsistent or conflicts with the content described in the present disclosure, the use of the description, definition and/or terms of the present disclosure shall prevail.

Finally, it should be understood that the embodiments described in the present disclosure merely illustrates the principles of the embodiments of the present disclosure. Other modifications may be within the scope of the present disclosure. Accordingly, by way of example, and not limitation, alternative configurations of embodiments of the present disclosure may be considered to be consistent with the teachings of the present disclosure. Accordingly, the embodiments of the present disclosure are not limited to the embodiments explicitly introduced and described by the present disclosure.

1. A method of controlling a sphygmomanometer, comprising:
selecting a pulse wave controlling parameter, wherein the pulse wave controlling parameter includes at least one of an amplitude threshold, a time threshold, or a heart rate threshold;
setting an initial pulse wave controlling parameter;
performing pressurization of a sphygmomanometer;
determining at least one pulse wave based on the initial pulse wave controlling parameter during the pressurization;
determining a corrected initial pulse wave controlling parameter by correcting the initial pulse wave controlling parameter based on at least one pulse wave controlling parameter of the at least one pulse wave;
determining at least one subsequent pulse wave based on the corrected initial pulse wave controlling parameter;
further correcting the corrected initial pulse wave controlling parameter based on at least one pulse wave controlling parameter of the at least one subsequent pulse wave;
repeating above iterative process and continuously correcting the initial pulse wave controlling parameter;
extracting at least one effective pulse wave based on the initial pulse wave controlling parameter after the correction; and
generating a blood pressure measurement result based on a detection result of the at least one effective pulse wave.

2. The controlling method of claim 1, wherein the correcting the initial pulse wave controlling parameter includes:
identifying a first pulse wave and a second pulse wave, wherein amplitudes of the first pulse wave and the second pulse wave are greater than an initial amplitude threshold;
correcting the initial amplitude threshold based on the amplitude of the first pulse wave and the amplitude of the second pulse wave after an initial determination that the first pulse wave and the second pulse wave are eligible,
identifying a third pulse wave based on the corrected initial amplitude threshold, an initial heart rate threshold, or an initial time threshold;
further correcting the initial amplitude threshold based on an amplitude of the third pulse wave;
correcting the initial heart rate threshold or the initial time threshold based on a time interval between the third pulse wave and the second pulse wave; and
determining a subsequent pulse wave based on the corrected initial amplitude threshold, the corrected initial time threshold, or the corrected initial heart rate threshold.

3. The controlling method of claim 2, wherein
a condition for the initial determination that the first pulse wave and the second pulse wave are eligible includes that a time interval between the first pulse wave and the second pulse wave is within a range of the initial heart rate threshold or the initial time threshold; and
if the time interval between the first pulse wave and the second pulse wave is out of the range of the initial heart rate threshold or the initial time threshold, discarding the first pulse, and determining a subsequent pulse wave of an amplitude greater than the initial amplitude threshold until a time interval between two adjacent pulse waves is within the range of the initial heart rate threshold or the initial time threshold.

4. The controlling method of claim 2, wherein
the initial amplitude threshold is a pressure of 0.2 mmHg;
the initial heart rate threshold is a minimum of 30 times per minute and a maximum of 300 times per minute; or
the initial time threshold is a minimum of 0.2 s and a maximum of 2 s.

5. The controlling method of claim 1, further comprising:
generating an effective pulse wave template based on the at least one effective pulse wave; and
filtering out noise interference in a pulse wave using the effective pulse wave template.

6. The controlling method of claim 5, wherein
if the noise interference is in a non-pulse wave portion of the effective pulse wave template, directly filtering out the noise interference; and
if the noise interference is in a pulse wave portion of the effective pulse wave template, performing fitting and compensation on a pulse wave at the pulse wave portion.

7. The controlling method of claim 6, wherein the performing fitting and compensation includes:
determining a changing trend of the at least one effective pulse wave over time based on the effective pulse wave template;
determining an inflection point in a curve of the changing trend;
determining an amplitude of the pulse wave based on an average of a sum of an amplitude of an effective pulse wave before the pulse wave and an effective pulse wave after the pulse wave in the effective pulse wave template;
determining a pulse wave time based on the effective pulse wave template; and
determining the pulse wave based on the pulse wave time, the amplitude of the pulse wave, and a position of the inflection point by a curve fitting technique using ordinary least squares.

8. The controlling method of claim 1, wherein the generating a blood pressure measurement result based on a detection result of the at least one effective pulse wave includes:
determining an amplitude of a pulse wave based on the initial pulse wave controlling parameter after the correction by:
determining a minimum value before the pulse wave; and
designating a maximum value among a plurality of sample points after a start point as a maximum value of the pulse wave, the start point corresponding to a first maximum value after the minimum value.

9. The controlling method of claim 8, wherein a count of the plurality of sampling points relates to a heart rate, a time interval of two adjacent sampling points of the plurality of sampling points being 1 millisecond.

10. The controlling method of claim 1, further comprising:
generating a final pressure for when the pressurization is to be ended based on the detection result of the at least one effective pulse wave, wherein the pressurization of the sphygmomanometer is ended if a pressure of the sphygmomanometer reaches the final pressure.

11. The controlling method of claim 10, wherein the generating a final pressure includes:
extracting an amplitude of each of the at least one effective pulse wave;
comparing at least one rising or declining trend of at least one amplitude of the at least one effective pulse wave;
determining a peak value of the at least one amplitude; and
determining the final pressure according to formula below:
P=Kx(Hr×m+Mp)
where K is a linear correlation coefficient of the pressurization in a macroscopic scale, Hr is a time interval between two adjacent effective pulse waves, m is a systolic pressure coefficient, and Mp is a time coordinate corresponding to the peak value of the at least one amplitude.

12. The controlling method of claim 1, further comprising:
controlling a pressurization speed by:
presetting a relationship between pressure and time, wherein each of pressures at different time points is determined based on the relationship and designated as a preset pressure, respectively;
detecting an actual pressure of the sphygmomanometer during the pressurization;
correcting the preset pressure based on the actual pressure; and
adjusting the pressurization speed of the sphygmomanometer based on the corrected preset pressure.

13. The controlling method of claim 12, wherein the adjusting the pressurization speed of the sphygmomanometer based on the corrected preset pressure includes:
comparing the actual pressure with the preset pressure;
if the actual pressure is greater than the preset pressure, decreasing the pressurization speed;
if the actual pressure is smaller than the preset pressure, increasing the pressurization speed; or
if the actual pressure is equal to the preset pressure, maintaining the pressurization speed.

14. The controlling method of claim 13, wherein the increasing or decreasing the pressurization speed relates to a difference between the actual pressure and the preset pressure.

15. The controlling method of claim 12, wherein the pressurization speed is adjusted using proportional adjustment in a proportional-integral-derivative (PID) control algorithm.

16. The controlling method of claim 12, wherein the preset relationship between pressure and time is a linear relationship.

17. A sphygmomanometer comprising:
a cuff configured to wind around a region to be measured;
an electric machine configured to inflate air into the cuff for pressurization:
and a processor, wherein the processor is configured to:
select a pulse wave controlling parameter and set an initial pulse wave controlling parameter, wherein the pulse wave controlling parameter includes at least one of an amplitude threshold, a time threshold, or a heart rate threshold;
continuously correct the initial pulse wave controlling parameter during the pressurization of the electric machine based on the initial pulse wave controlling parameter and a pulse wave determined based on the initial pulse wave controlling parameter, and extract at least one effective pulse wave based on the initial pulse wave controlling parameter after the correction; and
generate a blood pressure measurement result based on a detection result of the at least one effective pulse wave.

18. The sphygmomanometer of claim 17, further comprising:
a pressure detection component configured to detect an actual pressure in the cuff during the pressurization; and
an electric machine control component configured to adjust a pressurization speed of the electric machine, wherein
the electric machine control component adjusts the pressurization speed of the electric machine based on the actual pressure detected by the pressure detection component.

19. The sphygmomanometer of claim 18, wherein
the processor is further configured to determine a signal for ending the pressurization based on the at least one effective pulse wave extracted by the processor, and determines a final pressure; and
the electric machine control component is configured to control the electric machine to perform the pressurization and stop the pressurization if a pressure reaches the final pressure.

20. A method for detecting an effective pulse wave, comprising:
selecting a pulse wave controlling parameter, wherein the pulse wave controlling parameter includes at least one of an amplitude threshold, a time threshold, or a heart rate threshold;
setting an initial pulse wave controlling parameter;
determining at least one pulse wave based on the initial pulse wave controlling parameter:
determining a corrected initial pulse wave controlling parameter by correcting the initial pulse wave controlling parameter based on at least one pulse wave controlling parameter of the at least one pulse wave;
determining at least one subsequent pulse wave based on the corrected initial pulse wave controlling parameter;
further correcting the corrected initial pulse wave controlling parameter based on at least one pulse wave controlling parameter of the at least one subsequent pulse wave;
repeating above iterative process and continuously correcting the initial pulse wave controlling parameter; and
extracting at least one effective pulse wave based on the initial pulse wave controlling parameter after the correction.
